(19) United States
(12) Patent Application Publication
(10) Pub. No.: US 2026/0240547 A1
(43) Pub. Date: Aug. 20, 2026

(54) EMBOLIZATION DEVICE

(71) Applicant: MERIL LIFE SCIENCES PVT LTD, Vapi (IN)

(72) Inventors: PRAMOD KUMAR MINOCHA, Vapi (IN); DEVESHKUMAR MAHENDRALAL KOTHWALA, Surat (IN); MAHOMADOVESH MAHOMADYASIN DURANI, Taypi (IN); AAKIL IMTIYAZ, Valsad (IN)

(73) Assignee: MERIL LIFE SCIENCES PVT LTD, Vapi (IN)

(21) Appl. No.: 19/153,860

(22) PCT Filed: Jan. 17, 2024
(86) PCT No.: PCT/IN2024/050042

§ 371 Date: Aug. 5, 2025

(30) Foreign Application Priority Data
Feb. 6, 2023 (IN) 202321007367

(51) Int. Cl. A61B 17/12

(52) U.S. Cl. CPC A61B 17/12168; A61B 17/12031; A61B 17/12113; A61B 2017/12095

(57) ABSTRACT

An embolization device having a proximal lobe and a distal lobe connected by a bridge is disclosed. The proximal lobe has a braided mesh structure with a gradually increasing diameter from a first proximal end to a first distal end. The braided mesh structure is made from a plurality of wires and includes narrow pores to completely cut-off blood supply at an implantation site. The distal lobe is a braided mesh structure that extends from a second proximal end to a second distal end. The second distal end has a concave configuration which reduces overall length of the device thereby enhancing applicability of the device to varied implantation sites. The distal lobe includes a larger surface area than the proximal lobe thereby inhibiting chances of migration of the device and recanalization of the vessel.

### FIELD OF INVENTION

The present invention relates to a medical device. More specifically, the present invention relates to an embolization device for blocking a blood vessel.

### BACKGROUND OF THE INVENTION

Embolization corresponds to lodging of an embolus within a bloodstream. Embolization may be of natural origin or may be induced artificially as a treatment. Artificially induced or therapeutic embolization is performed as a hemostatic treatment for bleeding or as a treatment for some types of cancer by deliberately blocking blood vessels to starve the tumor cells.

One of the ways to artificially induce embolization is the use of an embolization device/occluder. An embolization device is a bio-medical device that creates an occlusion in the vasculature to obstruct the pathway of blood flow. The embolization device fits within a targeted blood vessel and totally cuts off the pathway of blood flow thereby helping to treat various conditions including aneurysm, left atrial appendage, atrial septal defect, fistulas, patent foramen ovule, patent ductus arteriosus vessel shutdown or occlusive purposes in the peripheral vasculature.

Ever since the advent of embolization devices, many structures of embolization devices have been devised. However, none of the existing embolization devices have been effective enough to fix firmly at an implantation site and occlude blood vessels by introducing an emboli. For example, the patent publication U.S. Pat. No. 10,470,773 discloses a vessel occluder that includes a singular lobe. The single lobed occluder plays both roles i.e., fixation of the occluder at the implantation site and embolization to completely cut-off blood flow at the implantation site. However, due to the single lobe construction, the device of this publication takes a lot of time to completely cut-off the blood flow at the implantation site thereby failing to provide instant hemostatic treatment to a patient. Further, such a device is also associated with issues relating to positional controllability as owing to the structure of the device, an operator does not have enough grip of the device at the time of implantation.

Moreover, embolization devices having multiple-lobes are also conventionally known. For example, the embolization devices disclosed in patent publications U.S. Pat. Nos. 10,624,619 and 8,313,505 include multi-layered braided formation having a dual lobe structure. The dual lobe structure is in the form of dumbbell shape having two similar parts at both proximal and distal ends that expand circumferentially. However, the presence of lobes having same shapes as included in the devices of the aforesaid publications is associated with various challenges. The most crucial one being incapacity to completely cut-off the blood flow at the implantation site and high chances of migration of the device leading to cardiac failure and pulmonary vascular disease.

Further, there are few conventional embolization devices having more than two lobes. However, such devices having multiple lobes typically require larger french size of the catheters for delivery. The use of larger size of catheters makes it very difficult for delivery of embolization devices especially in infants with congenital abnormalities. Thus, limiting the applicability of the existing embolization devices. Also, the multi-lobular structure of conventional devices increases the length of the device which in turn restricts the deployment of the device to limited vessels.

Therefore, an embolization device that addresses the aforesaid drawbacks is required to be devised.

### SUMMARY OF THE INVENTION

Particular embodiments of the present disclosure are described herein below with reference to the accompanying drawings, however, it is to be understood that the disclosed embodiments are mere examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The present invention relates to an embolization device (or device). The device includes a binary lobe structure having a proximal lobe and a distal lobe connected by a bridge is disclosed. The proximal lobe has a braided mesh structure with a gradually increasing diameter from a first proximal end to a first distal end (or gradually decreasing diameter from a first distal end to a first proximal end. The braided mesh structure is made from a plurality of wires and includes narrow pores to completely cut-off blood supply at an implantation site.

The distal lobe is a braided mesh structure that extends from a second proximal end to a second distal end. The second distal end has a concave configuration which reduces overall length of the device thereby enhancing applicability of the device to varied implantation sites. The distal lobe includes a larger surface area than the proximal lobe thereby inhibiting chances of migration of the device and recanalization of the vessel.

The foregoing features and other features as well as the advantages of the invention will become more apparent from the following detailed description, which proceeds with reference to the accompanying figures.

### BRIEF DESCRIPTION OF ACCOMPANYING DRAWINGS

The summary above, as well as the following detailed description of illustrative embodiments, is better understood when read in conjunction with the apportioned drawings. For the purpose of illustrating the present disclosure, exemplary constructions of the disclosure are shown in the drawings. However, the disclosure is not limited to specific methods and instrumentality disclosed herein. Moreover, those in the art will understand that the drawings are not to scale.

FIG. 1a depicts an embolization device 100 in delivery configuration in accordance with an embodiment of the present invention.

FIG. 1b depicts the embolization device 100 in deployed configuration in accordance with an embodiment of the present invention.

FIG. 2 depicts the embolization device 100 in accordance with an embodiment of the present invention.

FIG. 2a depicts a distal portion of the embolization device 100 in accordance with an embodiment of the present invention.

FIG. 2b shows a jacket 105 of the embolization device 100 in accordance with an embodiment of the present invention.

FIG. 3 shows the embolization device 100 connected to a delivery wire ‘w’ and a delivery catheter 200 in accordance with an embodiment of the present invention.

FIG. 3a illustrates the delivery wire ‘w’ in accordance with an embodiment of the present invention.

FIG. 3b shows the delivery wire ‘w’ in accordance with an embodiment of the present invention.

FIG. 4 indicates a process for manufacturing the embolization device 100 in accordance with an embodiment of the present invention.

FIG. 4a depicts a mold 300 used for shape setting in accordance with an embodiment of the present invention.

### DETAILED DESCRIPTION OF THE DRAWINGS

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like. Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although the operations of exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

The present invention discloses an embolization device that may be implanted at an implantation site in the form of a vessel segment located within a blood vessel in a patient's body. The embolization device of the present invention helps to obstruct pathway of blood flow at the implantation site thereby treating a pre-defined vascular defect. The embolization device of the present invention is capable of effectively treating arterial venous malformation, aneurysms, fistulas and other vascular defects.

The embolization device of the present invention is a self-expandable device having two lobes i.e. a proximal lobe and a distal lobe disposed towards a proximal end and a distal end of the device, respectively. The proximal end of the device corresponds to the end that extends away from an operator while the distal end corresponds to the end positioned towards the operator and attached to a delivery wire.

The proximal and the distal lobes are separated by a bridge. The proximal and the distal lobes are two distinct structures that confer flexibility and infrangibility during post-deployment and/or pre-deployment process.

The embolization device is implanted in such a way that the proximal lobe faces the blood flow pathway i.e., positioned against the blood flow. The distal lobe faces away from the blood flow pathway i.e., positioned along the blood flow. Therefore, the proximal lobe helps to promote early embolization and the distal lobe helps to reduce migration of the embolization device from the implantation site and supports the device during as well as after implantation.

Each of the proximal and the distal lobes includes a braided mesh structure. The mesh structures of the proximal and distal lobes include a pre-defined wire density and wire diameter to impart flexibility to the device and also allow the device to be easily pushed with required radial strength during and after the implantation.

The proximal lobe includes a conical structure made of a dense mesh structure with gradually increasing diameter and pore size. Densification of the braided mesh structure of the proximal lobe leads to reduced porosity which helps to completely shut-off the pathway of blood at the implantation site.

On the other hand, the mesh structure of the distal lobe is cylindrical in shape and helps to achieve better engagement with the vessel segment at the implantation site, thereby minimizing the possibility of migration of the device from the implantation site.

A polymer membrane is disposed inside the distal lobe which provides rapid embolization as compared to conventional systems. The polymer membrane helps to reduce and mitigate any chance of blood flow remaining after occlusion by the proximal lobe. Further, the embolization device has two or more radiopaque markers on both lobes that help in positioning and observation of the device during and after deployment procedure. Owing to the above structure, the embolization device of the present invention includes more surface contact area than the conventional systems which provides more grip of the embolization device at the time of implantation hence, overcoming the issues related to positional controllability with improved flexibility and controlled delivery.

The embolization device can be delivered using a small French size of the delivery catheter (5 to 9 Fr) thereby eliminating existing issues of applicability of the embolization devices in infants or limited vessels.

The embolization device of the present invention is constructed using a pre-defined method that provides adequate mechanical properties to the embolization device such as higher flexibility, kink resistance, low crimp profile, easy deployment, leak proof, etc.

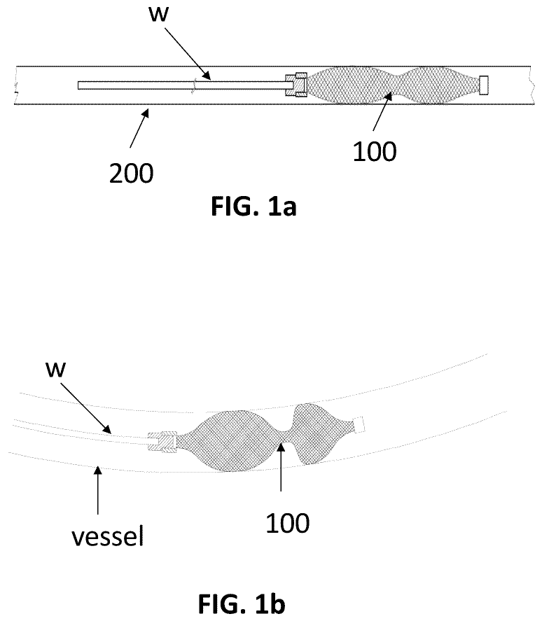

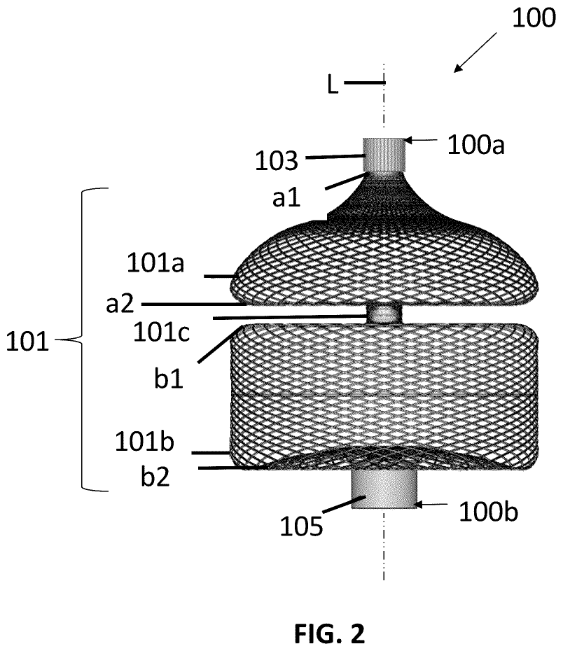

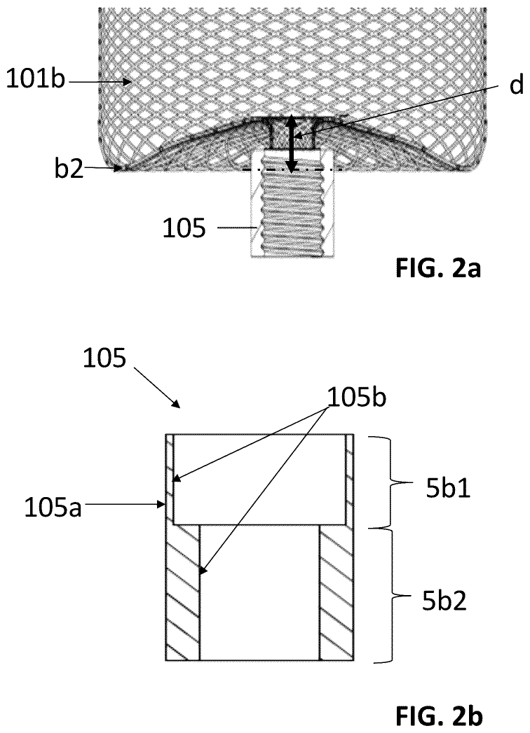

Now referring to figures, FIGS. 1a-1b and 2 disclose an embolization device 100 (or device 100) of the present invention. The embolization device 100 is a self-expandable device having a delivery configuration (FIG. 1a) and a deployed configuration (FIG. 1b). The delivery configuration corresponds to a configuration in which the embolization device 100 is in a compressed state for delivery at an implantation site. In the compressed state, the embolization device 100 is in an almost linear configuration and disposed inside a delivery catheter 200 as shown in FIG. 1a.

The deployed configuration corresponds to a configuration in which the embolization device 100 is in an expanded state and implanted at the implantation site. In the deployed configuration, the embolization device 100 expands from the almost linear configuration to a three-dimensional expanded configuration as illustrated in FIG. 1b. The embolization device 100 in deployed configuration, blocks/restricts the flow of blood through the blood vessel at the implantation site. The blood vessel of the implantation site may include a peripheral artery, a pulmonary artery, a splenic artery, a portal vein, an internal iliac artery, an arterio-venous malfunctions, an arterio-venous fistula, etc.

The embolization device 100 is deployed at the implantation site such that a longitudinal axis of the embolization device 100 coincides with an axis of a vessel segment of the blood vessel in which the embolization device 100 is being inserted.

In order to strongly engage a lumen of the vessel segment of the blood vessel at the implantation site, the embolization device 100 includes a maximum expanded diameter that is slightly more (approximately 30 to 50%) than the diameter of the lumen of the vessel segment of the blood vessel at the implantation site. Such selection of the maximum expanded diameter of the embolization device 100 helps to position the embolization device 100 securely against the lumen of the vessel segment of the implantation site. At the same time, the maximum expanded diameter of the device 100 so optimized does not cause any damage to an inner lining of the vessel or does not produce any bulge at an outer lining of the vessel.

The embolization device 100 is desirably longer along its axis than its maximum expanded diameter as shown in FIG. 1b. These dimensions of the device 100 substantially prevent the embolization device 100 from turning within the lumen at any angle with respect to the longitudinal axis thereby preventing the embolization device 100 from dislodgement along the vessel segment of the implantation site. The inability of the embolization device 100 to turn over helps to keep the embolization device 100 deployed precisely at the implantation site.

As seen from FIG. 1b, when deployed, the embolization device 100 engages the lumen of the vessel segment of the implantation site at two spaced apart locations. The said positioning is a resultant of the structure of the device 100.

The embolization device 100 extends from a proximal end 100a to a distal end 100b of the device 100 thus, defining the longitudinal axis ‘L’ of the embolization device 100. The embolization device 100 includes various components that are aligned with the longitudinal axis ‘L’. The embolization device 100 may include one or more of, a body 101, a proximal tube 103 and a distal tube (not shown) covered with a jacket 105.

The body 101 of the embolization device 100 is in the form of a hollow structure defined by at least two lobes and a bridge 101c. Each of the lobes of the present invention is in the form of a braided mesh structure made of a plurality of wires. The braided mesh structure significantly increases the density of wires thereby reducing stiffness in the embolization device 100 and confers an ability to attain a low outer diameter whenever the embolization device 100 is longitudinally stretched.

The mesh structures of the lobes include a pre-defined wire density and wire diameter that is selected to impart flexibility to the device 100 and also allow the device 100 to be easily pushed with required radial strength during and after the implantation.

The mesh structures of the lobes may be formed by a bio-compatible and/or bio-degradable material such as, without limitation, nitinol, poly-L-lactide (PLLA), etc. In an embodiment, the mesh structures of the lobes are made of nitinol.

The lobes may be structurally (i.e., shape and dimensions) same or different from each other. In an embodiment, the lobes are distinct, different from each other.

As an exemplary embodiment, the embolization device 100 includes a binary lobe structure having a proximal lobe 101a and a distal lobe 101b. As the name suggests, the proximal lobe 101a is disposed at the proximal end 100a of the device 100 while the distal lobe 101b is disposed at the distal end 100b of the device 100.

The proximal and distal lobes 101a and 101b, respectively, have different shapes and dimensions. The aforesaid binary lobe structure provides flexibility and mechanical strength to the embolization device 100 which effectively resists flow of blood inside a bulged capillary vessel of the implantation site. Owing to the presence of a binary lobe structure having distinct shape and dimensions, the issues relating to migration and complete cut-off of the blood flow are individually addressed via each of the two lobes (described below in detail).

In an embodiment as shown in FIG. 2, the proximal lobe 101a includes a conical shaped braided mesh structure extending from a first proximal end a1 to a first distal end a2. It should be noted that though the present invention is explained by way of a conical proximal lobe 101a, the possibility of other shapes such as spherical, concave, flattened oval, etc., is also within the scope of the present invention.

The conical shape of the proximal lobe 101a resists the blood flow inside the blood vessel of the implantation site and flips the blood to another capillary vessel.

Owing to the conical shape, the proximal lobe 101a includes gradually decreasing diameter from the first distal end a2 to the first proximal end a1 (or gradually increasing diameter from the first proximal end a1 to the first distal end a2). In an embodiment, the diameter of the proximal lobe 101a may range between 4 mm to 20 mm. The proximal lobe 101a includes a pre-defined length ranging between 4 mm to 9 mm.

The mesh structure of the proximal lobe 101a may be made from a plurality of braided wires in an arrangement of coil formation. In an embodiment, the proximal lobe 101a is made up of multiple layers of braided wires that result in formation of narrow pores of variable sizes. The pore size of the braided mesh structure may gradually decrease from the first distal end a2 to the first proximal end a1 and may range between 1-200 microns. The presence of narrow pores of variable sizes helps to shunt blood to another capillary vessel to completely cut-off blood supply at the implantation site and also prevents dislodging of the embolization device 100. Hence, the narrow pores of the proximal lobe 101a resist blood flow and improve filter property of the embolization device 100. Further, the narrow pores of the proximal lobe 101a help to create a wall against the forces created by the blood flow at the implantation site.

The first proximal end a1 may be positioned adjacent to the proximal tube 103 that marks the proximal end 100a of the device 100. The first distal end a2 may be placed adjacent to the bridge 101c facing the distal lobe 101b.

As an exemplary embodiment depicted in FIG. 2, the distal lobe 101b has a cylindrical shaped braided mesh structure. However, it should be noted that the possibility of having a distal lobe 101b of other shapes, such as spherical, concave, flattened oval, etc., is also within the scope of the present invention.

The cylindrical shape of the distal lobe 101b allows the embolization device 100 to properly connect to the lumen of the vessel segment at the implantation site and also absorbs forces which are created by the proximal end 100a of the embolization device 100.

Also, as an attribute of the cylindrical shape, the distal lobe 101b has a larger surface area than the proximal lobe 101a. Owing to the larger surface area of the distal lobe 101b, the embolization device 100 is able to fit well within the vessel segment of the implantation site thereby inhibiting the chances of migration and recanalization of the embolization device 100.

The distal lobe 101b may include a uniform diameter and a pre-defined length. The diameter of the distal lobe 101b may range between 4 mm to 20 mm. The distal lobe 101b may be expandable up to 4 mm to 9 mm. The length of the distal lobe 101b may be equal to or slightly more than the length of the proximal lobe 101a. The length of the distal lobe 101b may range between 4 mm to 9 mm. The nearly equal lengths of both the proximal lobe 101a and the distal lobe 101b facilitate easy molding of the device 100 at the time of manufacturing of the device 100.

The distal lobe 101b extends from a second proximal end b1 to a second distal end b2. The second proximal end b1 is placed adjacent to the bridge 101c and faces the first distal end a2 of the proximal lobe 101a. The second distal end b2 of the distal lobe 101b is placed adjacent to the jacket 105.

In an embodiment as shown in FIG. 2a, the second proximal end b1 is flat while the second distal end b2 of the distal lobe 101b includes a cupped or concave configuration. The cupped or concave configuration of the second distal end b2 helps in reducing overall length of the device 100 thereby providing a larger range of blood vessels in which the device 100 can be implanted as the device 100 can be easily implanted at the edge of the blood vessel.

Owing to the aforesaid cupped configuration, the distal lobe 101b at the second distal end b2 includes a dip with a depth ‘d’ ranging between 0.1 mm to 2 mm.

The distal lobe 101b may house a flexible membrane (not shown). The flexible membrane may help to initiate the endothelialization process for embolization and complete (full) embolization of blood flow. The flexible membrane may be made of polyethylene terephthalate (PET), expanded polytetrafluoroethylene (ePTFE), polyurethanes, metallic materials, extracellular matrix, synthetic bioabsorbable polymeric scaffolds, etc.). In an embodiment, the flexible membrane is composed of PET.

The dimensions of the flexible membrane may be dependent upon the dimensions of the distal lobe 101b. For example, the diameter of the flexible membrane is same as the diameter of the distal lobe 101b. The flexible membrane may include a pre-defined thickness ranging between 10-150 μm. The weight of flexible membrane may range between 10 GSM to 40 GSM.

The flexible membrane may be capable of expansion and contraction depending upon the configuration of the embolization device 100. For example, as the embolization device 100 expands from the delivery configuration to the deployed configuration, the flexible membrane also tends to expand. In an embodiment, the flexible membrane expands in a direction that is non-perpendicular to the longitudinal axis ‘L’ of the embolization device 100. In an alternate embodiment, the flexible membrane expands circularly, which is substantially perpendicular to the longitudinal axis ‘L’ of the embolization device 100.

The distance separating the above-described proximal and distal lobes 101a, 101b may vary depending upon the size of the blood vessel (implantation site) where the embolization device 100 is to be deployed.

In addition to the above, one or more radiopaque markers may be placed either on the proximal and/or distal lobes 101a, 101b to help a physician orient the embolization device 100 efficiently. The radiopaque markers may be in the form of radiopaque platinum wires or platinum iridium markers attached to the mesh structures of the proximal/distal lobes 101a, 101b in such manner that the radiopaque markers do not impede braid expansion or contraction. Other materials such as stainless steel, nitinol, or tantalum may also be used.

The above described proximal and distal lobes 101a, 101b are connected using the bridge 101c. The bridge 101c extends from the first distal end a2 of the proximal lobe 101a to the second proximal end b1 of the distal lobe 101b. The bridge 101c is in the form of a short cylindrical section as shown in FIG. 2. However, other shapes and structures of the bridge 101c are also within the scope of the present invention. The bridge 101c plays a major role in contraction and expansion of the embolization device 100 and also imparts mechanical strength and flexibility to the proximal and distal lobes 101a, 101b. Also, as the bridge 101c connects the proximal and distal lobes 101a, 101b, the bridge 101c helps to relieve pressure at the time of loading the embolization device 100 inside a loader. The bridge 101c provided vacant time during loading, which helps in expansion and contraction of the device 100 during loading and unloading, thus, reducing manual effort of loading.

As an exemplary embodiment shown in FIG. 1, the proximal tube 103 is provided at the proximal end 100a of the embolization device 100. The proximal tube 103 helps to hold and secure/seal the free ends of the braided mesh structure of the proximal lobe 101a that are formed at the time of braiding. Hence, the proximal tube 103 and the distal tube are useful to prevent the free ends of the braided mesh structure from unraveling at either end of the body 101, thus, maintaining the shape of the body 101.

The proximal tube 103 may be made of radiopaque material such as stainless steel (SS316L grade), nitinol, platinum, platinum-iridium, tantalum. In an embodiment, the proximal tube 103 is made of SS316L. The proximal tube 103 also works as a marker during surgery to identify the position of the device 100 during fluoroscopy. The proximal tube 103 includes pre-defined shape and dimensions. The cross-section shape of the proximal tube 103 may be circular, polygonal, etc. In an embodiment, the proximal tube 103 includes a cylindrical shape having a circular cross-section. The dimensions of the proximal tube 103 may depend upon the number of wires and diameters of each wire that is used in the device 100.

The distal tube may be structurally (including shape and dimensions) and functionally same as the proximal tube 103 and hence, helps to hold and secure/seal the free ends of the of the braided mesh structure of the distal lobe 101b that are formed at the time of braiding. The same way as the proximal tube 103, the distal tube may also be made of radiopaque material such as stainless steel, nitinol, platinum, platinum-iridium, tantalum. In an embodiment, the distal tube is made of SS316L. The distal tube (not visible in figures) is covered with the jacket 105. The proximal tube 103 and distal tube may have inner diameter 0.8 mm to 1.2 mm and outer diameter 1.0 mm to 1.5 mm while length 0.8 mm to 1 mm.

The jacket 105 is provided with the distal tube to help in attachment of the device 100 with the delivery wire ‘w’ for delivery and deployment procedure.

The jacket 105 may be in the form of a cap and is welded or crimped over the distal tube. However, other structural embodiments of the jacket 105 are also within the scope of the present invention.

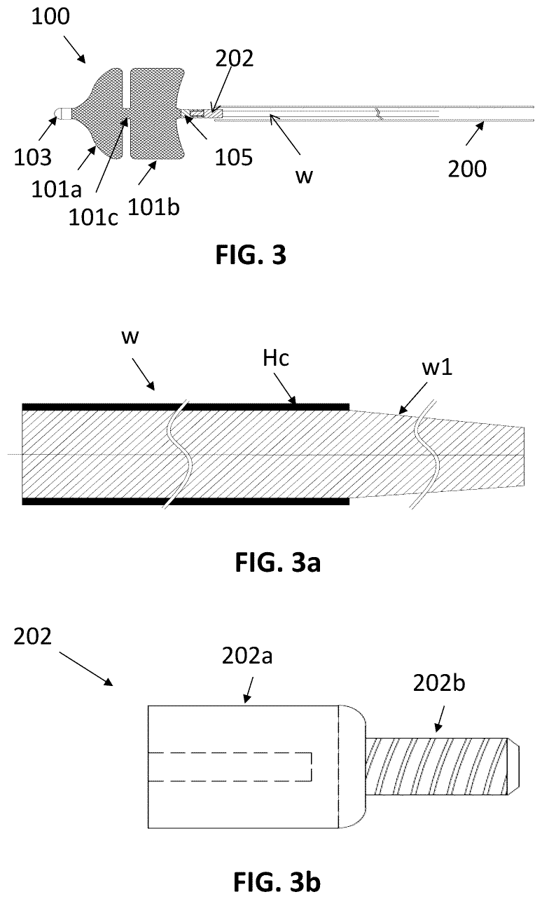

FIG. 2b depicts an exemplary structure of the jacket 105. The jacket 105 includes a uniform outer surface 105a and an indented inner surface 105b. The indented inner surface 105b of the jacket 105 defines a first cavity 5b1 and a second cavity 5b2. The first cavity 5b1 is structured to fit over the distal tube. The second cavity 5b2 is used for engaging the delivery wire ‘w’ via a delivery wire screw (202 shown in FIGS. 3 and 3b). In an embodiment as shown in FIG. 2b, the second cavity 5b2 includes threaded walls to engage the delivery wire screw 202 (described below in detail).

Therefore, the dimensions of the first cavity 5b1 may be dependent upon the dimensions of the distal tube while the dimensions of the second cavity 5b2 may be dependent upon the dimensions of the delivery wire screw 202.

The foregoing embolization device 100 may be delivered using the delivery catheter 200 and the delivery wire ‘w’. The delivery catheter 200 navigates the embolization device 100 through the vasculature of the patient's body for delivery at the implantation site. The delivery catheter 200 may be operated using a remote which is located outside the patient's body. However, other means of operation of the delivery catheter 200 are also within the scope of the present invention.

The delivery wire ‘w’ helps to guide the embolization device 100 and the delivery catheter 200 through the vasculature to arrive at the implantation site. The delivery wire ‘w’ is connected to the device 100 via the delivery wire screw 202 as shown in FIG. 3.

The delivery wire ‘w’ may be in the form of an elongate flexible metal shaft or a braided tube. The delivery wire ‘w’ may be made of nitinol, medical grade low alloy stainless steel or any other biocompatible metals. In an embodiment, the delivery wire ‘w’ is in the form of a distally tapered nitinol wire as shown in FIG. 3a.

The delivery wire ‘w’ may be provided with a hydrophilic coating ‘Hc’. Such a coating provides extra lubricity to the delivery wire ‘w’ which helps minimizing flow resistance during delivery of the device 100. In an embodiment, the hydrophilic coating ‘Hc’ of polytetrafluoroethylene (PTFE) or Teflon is provided over the delivery wire ‘w’. This coating may be applied over the whole length of the delivery wire ‘w’ or a part of it. For example, for the purpose of retaining flexibility and ease of handling, a tapered portion ‘w1’ of the delivery wire ‘w’ is not coated with any hydrophilic coating ‘Hc’ as evident from FIG. 3a.

The tapered portion ‘w1’ of the delivery wire ‘w’ is structured to connect with the delivery wire screw 202.

An exemplary structure of the delivery wire screw 202 (or screw 202) is shown in FIG. 3b. As shown in FIG. 3b, the screw 202 is in the form of a hollow cap 202a having an extension 202b. The hollow cap 202a may house the tapered portion ‘w1’ of the delivery wire ‘w’. In an embodiment, the tapered portion ‘w1’ of the delivery wire ‘w’ is welded or crimped within the hollow cap 202a of the screw 202. The extension 202b is connected to the second cavity 5b2 of the jacket 105. In an embodiment, the extension 202b are threaded to engage with the second cavity 5b2 via a threaded connection. Such a threaded connection allows easy engagement and disengagement of the embolization device 100 with the delivery wire ‘w’.

For delivery of the device 100, the delivery catheter 200 housing the delivery wire ‘w’ connected with the device 100 is advanced to the implantation site through a sheath.

The delivery catheter 200 includes a provision for repositioning the embolization device 100 if it is determined that the embolization device 100 is not properly positioned within the shunt. When the embolization device 100 is deployed, the delivery catheter 200 retains the device 100 till the device 100 is properly positioned at the implantation site. The delivery wire ‘w’ may be rotated about its axis to unscrew the embolization device 100 from the delivery wire screw 202.

Achievement of proper positioning and placement of the embolization device 100 leads to formation of endocardial layer over the embolization device 100 thereby inhibiting the growth of bacterial endocarditic and thromboembolisms.

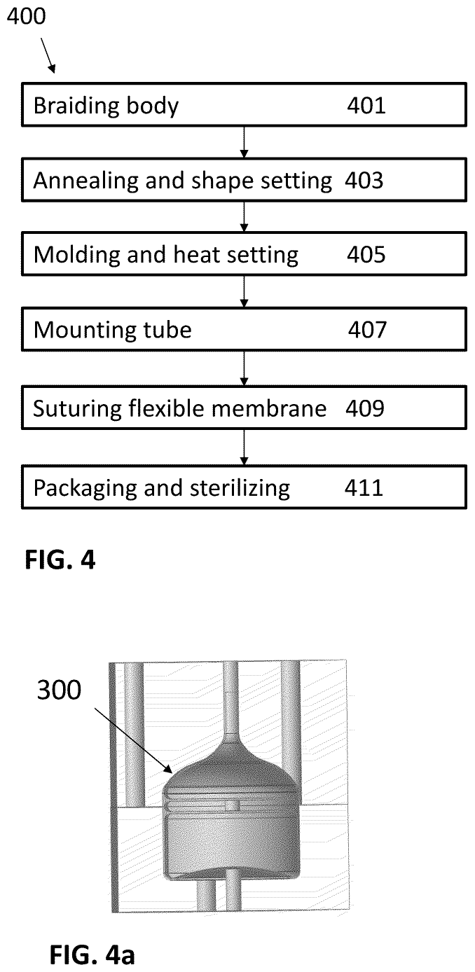

The above-described embolization device 100 is formed using a pre-defined methodology 400 as illustrated in FIG. 4. At step 401, the body 101 of the embolization device 100 is formed via filament braiding. In the filament braiding process, a plurality of wires is braided in filament form at a pre-defined angle. The wires may be made of a shape memory material such as nitinol, a nickel titanium alloy. The number of braided wires may range from 32 to 144, depending upon the desired characteristics of the particular device 100. As an exemplary embodiment, 72 nitinol wires are braided at an angle of 105 to 145 degrees. The diameter of the wires may range from 0.068-0.101 mm. In an embodiment, the diameter of the wires is 35 microns to 120 microns.

Each braid includes two sets of essentially parallel helical strands with one set of strands having clockwise direction and the other set of strands having an anti-clockwise direction.

A typical pitch angle may range from 30-70 degrees from the longitudinal axis of the braided tube (as braided relaxed tube prior to heat treatment). It should be noted that the pitch and the wire diameter are all variables that can be altered to change the shape and characteristics of the embolization device 100.

Therefore, at the end of step 401, a braided nitinol tube is generated using a braiding machine and is converted into spindle design in such a way that it reduces extra pressure on the wires.

At step 403, the braided tube is annealed and heat set in cylindrical shape at 505° C. for 5 minutes. The annealing performed at step 403 helps to obtain the device 100 having the desired shape.

At step 405, the annealed tube is molded and shape set. In order to shape set the braided tube, a mold 300 is used. The mold 300 used in the present invention is shown in FIG. 4a. The tube is placed over the mold 300 and heated at a pre-defined temperature for a pre-defined time.

The parameters for shape setting may be dependent upon the material of the wires used. For example, for nitinol braided tube, the parameters are 505° C. temperature and 5 minutes time duration.

After the heat treatment for shape setting, a molded tube is obtained and is removed from contact with the mold surface. The molded tube corresponds to the body 101 of the embolization device 100 having the proximal lobe 101a and the distal lobe 101b connected by the bridge 101c.

At step 407, the body 101 formed at step 405 is mounted over the proximal tube 103 and the distal tube. To avoid fatigue and abrasion of the wires, the diameter of the proximal tube 103 and the distal tube include some clearance. The clearance is maintained in a range of 10-30% of the diameter of the proximal tube 103 and the distal tube. Each of the proximal tube 103 and distal tube includes an outer diameter which is dependent on the size of the delivery catheter 200. To avoid scratching of an inner surface of the delivery catheter 200 by the embolization device 100, the inner diameter of the delivery catheter 200 is more than the outer diameter of the proximal tube 103 and the distal tube. The body 101 is coupled to the proximal tube 103 and the distal tube in a pre-defined manner. In an exemplary embodiment, the proximal tube 103 and the distal tube is manually mounted using forceps or an equivalent component. The ends of the proximal and distal lobes 101a and 101b may be encapsulated by a para film or a similar material.

Once the tube mounting is completed, the loose ends of the proximal and the distal lobes 101a and 101b are sealed using laser welding, spot welding or crimping. The distal tube towards the distal lobe 101b is provided with the jacket 105 and welded thereof.

At step 409, the flexible membrane is stitched to the distal lobe 101b. The polymeric fibers in the flexible membrane may include monofilament or multifilament yarns ranging from about 50-300 denier. The individual filaments may range from about 0.25-10 denier.

In an embodiment, a wax finished polyester suture is used to stitch the flexible membrane. The suture tightly bonds the flexible membrane over the embolization device 100. The size of the suture may range between 3/0 to 5/0 USP. The flexible membrane is stitched using two over two pattern of stitching.

Once the flexible membrane has been stitched, the embolization device 100 is formed.

At step 411, the embolization device 100 is packaged and sterilized followed by secondary and final packaging. In an embodiment, the embolization device 100 is sterilized using ETO sterilization.

In order to test the efficiency of the embolization device 100 as elaborated in the foregoing description, in-vitro testing was performed inside a silicon tube to study migration of the embolization device 100. The embolization device 100 of 14×14 mm was placed inside a 10 mm silicon tube. Using a peristaltic pump, the water was allowed to pass through the silicon tube at a flow rate of 79 ml/minute for 5 minutes. No migration of the embolization device 100 was observed at the flow rate of 79 ml/minute. The flow rate was gradually increased up to 120 ml/minute. Still, no migration of the embolization device 100 was found. Also, the embolization device 100 was found to completely cut-off the water flow through it at all the tested flow rates.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

We claim:

1. An embolization device (100) comprising:
a proximal lobe (101a) disposed at a proximal end (100a) of an embolization device (100), the proximal lobe (101a) having a braided mesh structure extending from a first proximal end (a1) to a first distal end (a2), wherein the proximal lobe (101a) having a gradually decreasing diameter from the first distal end (a2) to the first proximal end (a1), wherein the braided mesh structure being made from a plurality of braided wires that form narrow pores of variable sizes to completely cut-off blood supply at an implantation site;
a distal lobe (101b) disposed at a distal end (100b) of the embolization device (100), the distal lobe (101b) having a braided mesh structure extending from a second proximal end (b1) to a second distal end (b2); and
a bridge (101c) connecting the proximal lobe (101a) and the distal lobe (101b), wherein the bridge (101c) extending from the first distal end (a2) of the proximal lobe (101a) to the second proximal end (b1) of the distal lobe (101b),
wherein the distal lobe (101b) includes a larger surface area than the proximal lobe (101a).

2. The embolization device (100) as claimed in claim 1, wherein the proximal lobe (101a) is conical in shape.

3. The embolization device (100) as claimed in claim 1, wherein the narrow pores of the proximal lobe (101a) include gradually decreasing pore size ranging between 1 to 200 microns from the first distal end (a2) to the first proximal end (a1).

4. The embolization device (100) as claimed in claim 1, wherein the first proximal end (a1) of the proximal lobe (101a) is coupled to a proximal tube (103) for holding and securing the free ends of the braided mesh structure.

5. The embolization device (100) as claimed in claim 1, wherein the distal lobe (101b) is cylindrical in shape.

6. The embolization device (100) as claimed in claim 1, wherein the second distal end (b2) of the distal lobe (101b), includes a concave configuration having a depth ranging between 0.1 to 2 mm.

7. The embolization device (100) as claimed in claim 1, wherein the second distal end (b2) of the distal lobe (101b) is coupled to a distal tube for holding and securing the free ends of the braided mesh structure.

8. The embolization device (100) as claimed in claim 7, wherein the distal tube is covered with jacket (105) facilitating a for attachment of the embolization device (100) with a delivery wire (w) for delivery and deployment of the embolization device (100).

9. The embolization device (100) as claimed in claim 1, wherein the distal lobe (101b) includes a flexible membrane within the distal lobe (101b) which facilitates endothelialization and full embolization of blood flow.

10. The embolization device (100) as claimed in claim 9, wherein the flexible membrane is made from a material selected from polyethylene terephthalate (PET), expanded polytetrafluoroethylene (ePTFE), polyurethanes, metallic materials, extracellular matrix or synthetic bioabsorbable polymer.

11. The embolization device (100) as claimed in claim 1, wherein the proximal and distal lobes (101a, 101b) are provided with one or more radiopaque markers made from stainless steel, nitinol, platinum, platinum-iridium, or tantalum.
